(19) United States
(12) Patent Application Publication
(10) Pub. No.: US 2003/0181871 A1
(43) Pub. Date: Sep. 25, 2003

(54) Needle assembly

(75) Inventors: Bradley Wilkinson, North Haledon, NJ (US); Volker Niermann, Bound Brook, NJ (US); Jamieson Crawford, New York, NY (US)

Correspondence Address: THE WEBB LAW FIRM/BECTON, DICKINSON AND COMPANY, 700 KOPPERS BUILDING, 436 SEVENTH AVENUE, PITTSBURGH, PA, 15219, US

(73) Assignee: Becton, Dickinson and Company

(21) Appl. No.: 10/391,995

(22) Filed: Mar. 19, 2003

Related U.S. Application Data
(60) Provisional application No. 60/365,702, filed on Mar. 19, 2002.

(51) Int. Cl. A61M 5/178

(52) U.S. Cl. 604/263

(57) ABSTRACT

A shieldable needle assembly provided as part of a blood collection set includes a needle cannula having a proximal end and a distal end with a puncture tip. The needle assembly has a hub member supporting the proximal end of the needle cannula and a shield member having a housing defining a central bore, which is in axial alignment with the hub member. The shield member extends co-axially about the needle cannula and is movable between a retracted position in which the puncture tip of the needle cannula is exposed, and an extended position covering the puncture tip of the needle cannula. An extendable member connects the hub and shield members. A compression spring is further connected between the hub and shield members for moving the shield member to the extended position. A latch assembly provides releasable engagement between the hub and shield members.

### CROSS REFERENCE TO RELATED APPLICATIONS

This application claims the benefit of U.S. Provisional Application No. 60/365,702 filed Mar. 19, 2002.

### BACKGROUND OF THE INVENTION

1. Field of the Invention

The present invention relates to blood collection sets for safe and convenient handling and disposal of medical needles and, more particularly, a disposable blood collection set including a passively actuated shieldable needle assembly.

2. Description of Related Art

Disposable medical devices having medical needles are used for administering medication or withdrawing fluid from the body of a patient. Such disposable medical devices typically include blood collecting needles, fluid handling needles, and assemblies thereof. Current medical practice requires that fluid containers and needle assemblies used in such devices be inexpensive and readily disposable. Consequently, existing blood collection devices typically employ some form of durable, reusable holder on which detachable and disposable medical needles and fluid collection tubes may be mounted. A blood collection device of this nature may be assembled prior to use and then disassembled after use. Thus, these blood collection devices allow repeated use of a relatively expensive holder upon replacement of relatively inexpensive medical needles and/or fluid collection tubes. In addition to reducing the cost of collecting blood specimens, these blood collection devices help minimize the production of hazardous waste material.

A blood collection device or intravenous “IV” infusion device typically includes a needle cannula having a proximal end, a pointed distal end, and a lumen extending therebetween. The proximal end of the needle cannula is mounted in a plastic hub defining a central passage that communicates with the lumen extending through the needle cannula. A thin, flexible thermoplastic tube is connected to the hub and communicates with the lumen of the needle cannula. The end of the plastic tube remote from the needle cannula may include a fixture for connecting the needle cannula to a blood collection tube or other receptacle. The specific construction of the fixture will depend upon the characteristics of the receptacle to which the fixture is to be connected.

In order to reduce the risk of incurring an accidental needle-stick wound, protection of used needle cannulas becomes important. With concern about infection and transmission of diseases, methods and devices to enclose or cover the used needle cannula have become very important and in great demand in the medical field. For example, needle assemblies commonly employ a safety shield that can be moved into shielding engagement with a used needle cannula to minimize risk of an accidental needle-stick.

Some needle safety shields are referred to as “tip guards” and include a small rigid guard that may be telescoped along the length of the needle cannula and extended over the pointed distal end of the needle cannula for protection.

Such conventional tip guards may include some form of tether for limiting the travel of the tip guard to the length of the needle cannula. An example of the foregoing is disclosed by U.S. Pat. No. 5,176,655 to McCormick et al. The McCormick et al. patent discloses the use of flexible loop-like straps for limiting the distal movement of a tip guard.

Needle shields that incorporate movable tip guards are typically manually actuated. For example, U.S. Pat. Nos. Re 36,447 and Re 36,398, both to Byrne et al., disclose a safety device for a hypodermic needle that includes a plastic sheath, which is used to cover the puncture tip of the needle. The plastic sheath incorporates a thumb guard, which the user of the safety device may grasp to move the plastic sheath to a position covering the puncture tip of the needle cannula. U.S. Pat. No. 5,951,525 to Thorne et al. discloses a manually operated safety needle apparatus that includes two pairs of opposed legs adapted to move the tip guard of the apparatus to a position covering the used needle cannula. U.S. Pat. Nos. 5,562,637 and 5,562,636, both to Utterburg, disclose a rectangular needle protector sheath for use with a needle cannula that may be extended over the needle cannula after its use.

Other prior art devices, such as those disclosed by U.S. Pat. No. 5,290,264 to Utterberg and U.S. Pat. No. 5,192,275 to Burns provide “grippable” members attached to the tip guards to facilitate moving the tip guards to a position covering the puncture tip of a needle cannula. In addition to providing gripping members for moving the tip guards, prior art devices in this area often include flexible wings, which are used as means for securing the needle assemblies to the body of a patient during a medical procedure. Examples of “winged” needle assemblies may be found in U.S. Pat. No. 5,120,320 to Fayngold; and U.S. Pat. Nos. 5,154,699; 5,088,982; and 5,085,639 all to Ryan. Other prior art in this area includes U.S. Pat. Nos. 5,266,072 and 5,112,311, both to Utterberg et al., which also disclose guarded winged needle assemblies.

Conventional shields and tip guards, such as those discussed hereinabove, often further require extensive mechanics for positioning the shield or tip guard over the needle cannula. This results in complex arrangements that are costly to manufacture and assemble. Additionally, operation of the needle assemblies to move the tip guard into the proper position over the pointed distal end of the needle cannula requires substantial manual manipulation by the user of the device, exposing the user to potential needle-stick wounds.

In view of the foregoing, a need exists for a blood collection set including a shieldable needle assembly that achieves secure and effective shielding of a used needle cannula while requiring minimal manual input from the user of the needle assembly, and which is simple and inexpensive to manufacture.

### SUMMARY OF THE INVENTION

The present invention is directed to a blood collection set that includes a shieldable needle assembly. The blood collection set generally includes a fixture for connecting the blood collection set to a receptacle, a flexible tube connected to the fixture, and the shieldable needle assembly. The flexible tube has opposed first and second ends, with the first end of the flexible tube connected to the fixture. The needle assembly used in the blood collection set generally includes a needle cannula having a proximal end and a distal end with a puncture tip. The proximal end of the needle cannula is connected to a hub member, which supports the proximal end of the needle cannula. The distal end of the needle cannula projects outward from the hub member. The needle cannula also defines a lumen in fluid communication with the flexible tube and the fixture. The needle assembly further includes a shield member having a housing defining a central bore. The shield member is in axial alignment with the hub member and extends coaxially about the needle cannula. The shield member is movable between a retracted position in which the puncture tip of the needle is exposed, and an extended position in which the shield member covers the puncture tip of the needle cannula. An extendable member is connected between the hub member and the shield member. A compression spring is operatively connected between the hub member and the shield member. A latch assembly provides releasable engagement between the hub member and the shield member. When the latch assembly is in engagement between the hub member and the shield member, the shield member is in the retracted position. When the latch assembly is released of engagement between the hub member and the shield member, the shield member is moved to the extended position through forces exerted by the compression spring, thereby covering the puncture tip of the needle cannula.

The latch assembly may include a pivoting latch member located on the hub member or the shield member. The latch member is generally configured to releaseably engage with a catch member located on the other of the hub member and shield member. Thus, when the latch member is pivoted out of engagement with the catch member, the latch assembly is released of engagement between the hub member and the shield member, thereby causing the shield member to be moved to the extended position by the compression spring.

The shield member may include a pair of butterfly wings extending laterally from opposing sides of the housing of the shield member. The housing of the shield member may further include a dorsal wing located between the butterfly wings. Similary, the hub member may include a pair of butterfly wings, and a dorsal wing located between the butterfly wings.

The compression spring may be located co-axially about the needle cannula. Alternatively, the compression spring may be located adjacent the needle cannula, extending between the hub member and the shield member.

The shield member may further include a tip guard for protectively surrounding the puncture tip of the needle cannula when the shield member is moved to the extended position. Alternatively, the tip guard may be externally attached to the housing of the shield member.

The extendable member may include a pair of folding legs connected by a hinged knee joint. Preferably, one leg of the pair of legs is hingedly connected to the hub member and the other leg of the pair of legs is hingedly connected to the shield member. The knee joint may further include a fingerplate to enable the user to manipulate the assembly.

The needle assembly may include a locking assembly for locking the shield member in the extended position. The locking assembly may include a pair of locking members extending from the fingerplate toward the needle cannula and configured to engage the needle cannula such that when the shield member is moved to the extended position by the compression spring, the locking to secure the shield member in the extended position.

The needle assembly may include a second extendable member connecting the hub and shield members and located opposite from the first extendable member. Each of the extendable members may include a pair of folding legs connected by a hinged knee joint, with one leg of the pair of legs for each extendable member hingedly connected to the hub member and the other leg of the pair of legs for each extendable member hingedly connected to the shield member. The knee joint for each extendable member may include a fingerplate. A locking assembly may be provided for locking the shield member in the extended position, with the extendable members extending therebetween. The locking assembly may include a locking member extending from one of the fingerplates toward the opposing fingerplate. The locking member is preferably configured to engage a second locking member extending from the opposing fingerplate, such that when the shield member is moved to the extended position by the compression spring, the locking members engage to secure the shield member in the extended position.

The hub member and shield member may each include a pair of pivoting butterfly wings extending laterally from opposing sides thereof. The latch assembly may be located to provide releasable connections between the butterfly wings extending from the hub and shield members. For example, the latch assembly may include a pair of latch members located, respectively, on the wings extending from the shield member and be configured to releasably engage with a second pair of latch members located, respectively, on the wings extending from the hub member. When the respective pairs of latch members are engaged, the shield member is maintained in the retracted position. When one of the pairs of wings extending from the hub and shield members is pivoted, the engaged latch members are disengaged and the shield member is moved to the extended position by the compression spring, thereby covering the puncture tip of the needle cannula.

Furthermore, the present invention is directed to a method of automatically placing a shieldable needle assembly into a safety state in which a puncture tip of a needle cannula is covered by the needle assembly. The method may generally include the steps of: providing a shieldable needle assembly as generally described hereinabove; using the needle assembly in a medical procedure; and pivoting the latch member of the latch assembly out of engagement with the catch member of the latch assembly such that the shield member is moved to the extended position through the force of the compression spring, thereby covering the puncture tip of the needle cannula.

Further details and advantages of the present invention will become apparent from the following detailed description read in conjunction with the drawings.

### DESCRIPTION OF THE DRAWINGS

FIG. 1 is a perspective view of a blood collection set and a shieldable needle assembly in accordance with a first embodiment of the present invention showing the needle assembly in a retracted position;

FIG. 2 is a perspective view of the blood collection set of FIG. 1 showing the needle assembly in a partially extended position;

FIG. 3 is a perspective of the blood collection set of FIG. 1 showing the needle assembly in a fully extended and locked position;

FIG. 4 is a cross-sectional view taken along line IV-IV of FIG. 1;

FIG. 5 is a cross-sectional view taken along line V-V of FIG. 1;

FIG. 6 is a side view of a tip guard portion of the needle assembly of FIG. 1;

FIG. 7 is a side view of the tip guard portion of FIG. 6 showing the tip guard portion covering a distal end of the needle assembly;

FIG. 8 is a perspective view of the blood collection set and shieldable needle assembly in accordance with a second embodiment of the present invention showing the needle assembly in a retracted position;

FIG. 9 is a perspective view of the blood collection set of FIG. 8 showing the needle assembly in a partially extended position;

FIG. 10 is a cross-sectional view taken along line X-X of FIG. 8;

FIG. 11 is a perspective view of the blood collection set of FIG. 1 wherein the needle assembly incorporates a modified tip guard portion in accordance with the present invention;

FIG. 12 is a perspective view of the blood collection set of FIG. 11 showing the needle assembly in a partially extended position;

FIG. 13 is a cross-sectional view taken along line XIII-XIII in FIG. 11;

FIG. 14 is a cross-sectional view taken, along line XIV-XIV in FIG. 12;

FIG. 15 is a perspective view of the blood collection set and shieldable needle assembly in accordance with a third embodiment of the present invention showing the needle assembly in a retracted position;

FIG. 16 is a perspective view of the blood collection set of FIG. 15 showing the needle assembly in a partially extended position;

FIG. 17 is a cross-sectional view taken along line XVII-XVII in FIG. 15;

FIG. 18 is a cross-sectional view taken along line XVIII-XVIII in FIG. 15;

FIG. 19 is a perspective view of the shieldable needle assembly in accordance with a fourth embodiment of the present invention; and

FIG. 20 is a perspective view of the needle assembly of FIG. 19 including the modified tip guard portion of FIG. 11.

### DETAILED DESCRIPTION

Referring to the drawings in which like reference characters refer to like parts throughout the several views thereof, the present invention is generally described in terms of a blood collection set and related features, and encompasses such a blood collection set as well as a shieldable needle assembly for use in such a blood collection set.

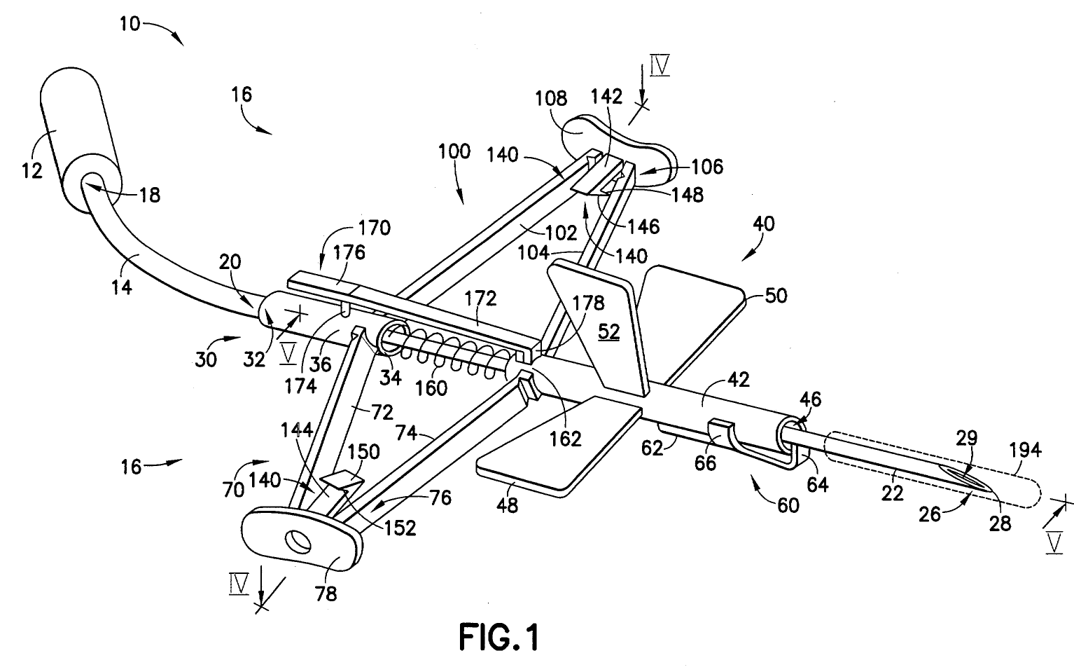

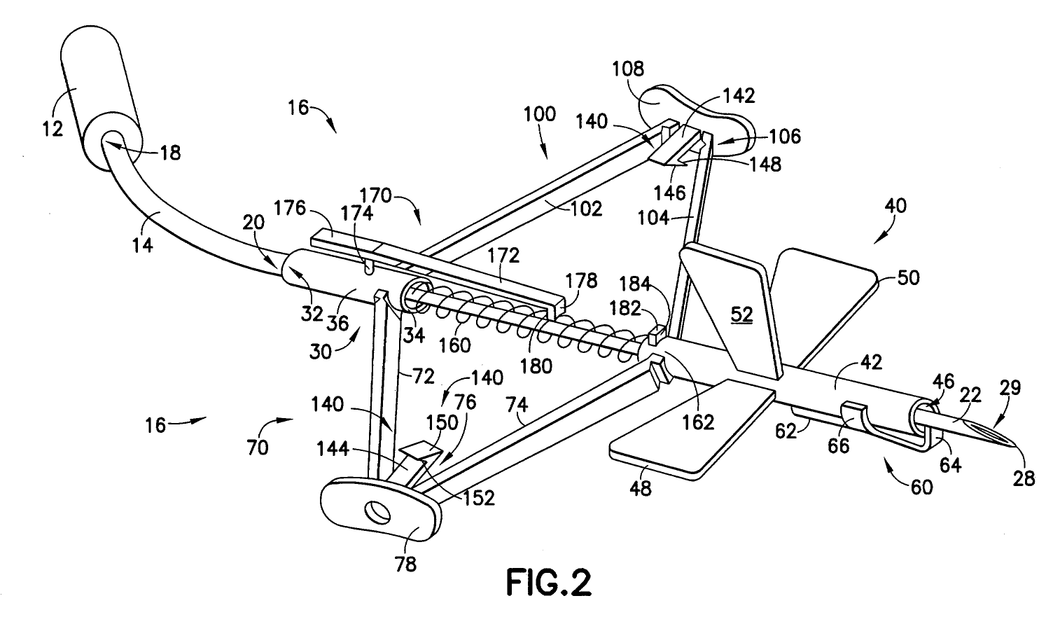

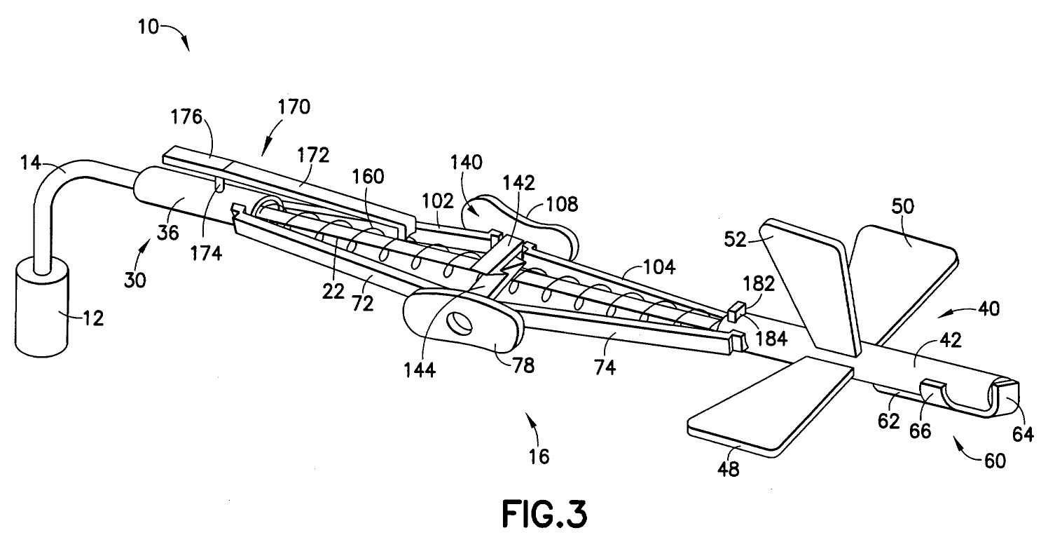

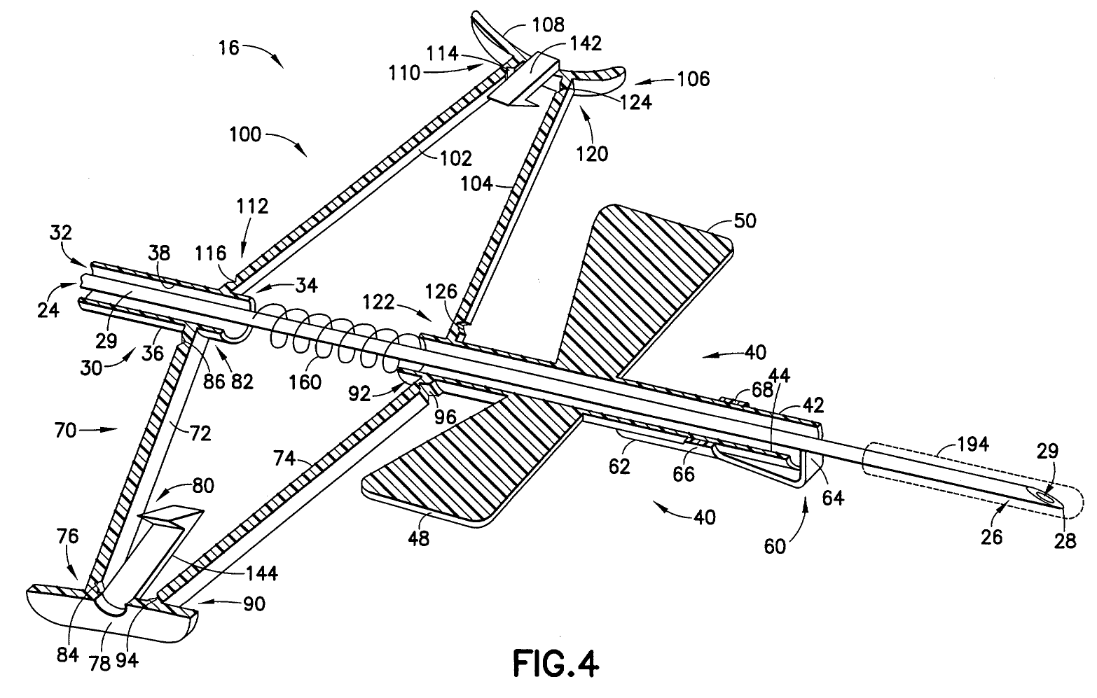

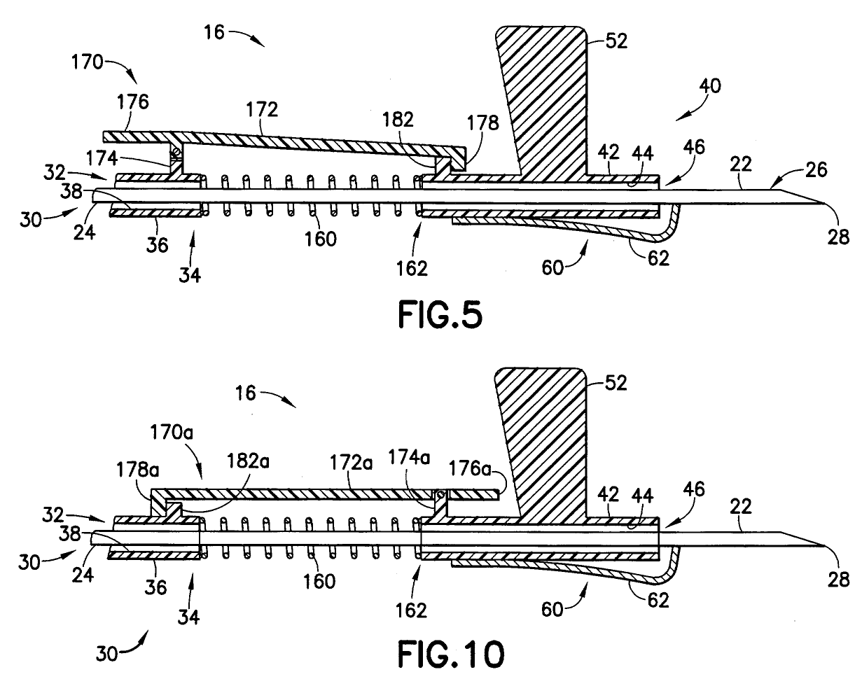

Referring to FIGS. 1-5, a blood collection set 10 in accordance with the present invention includes a fixture 12 for connecting the blood collection set 10 to a receptacle (not shown), a flexible tube 14, and a shieldable needle assembly 16. The flexible tube 14 has a first end 18 and a second end 20. The first end 18 of the flexible tube 14 is connected to the fixture 12. The first end 18 of the flexible tube 14 may be connected to the fixture 12 by means customary in the art.

The shieldable needle assembly 16 of the blood collection set 10 includes a needle cannula 22. The needle cannula 22 has a proximal end 24 and an opposing distal end 26. The needle cannula 22 defines a lumen 29 extending through the needle cannula 22 from the proximal end 24 to the distal end 26. The distal end 26 of the needle cannula 22 is beveled to define a sharp puncture tip 28, such as an intravenous (IV) puncture tip. The puncture tip 28 is provided for insertion into a patient's blood vessel, such as a vein, and is, therefore, designed to provide ease of insertion and minimal discomfort during venipuncture.

The shieldable needle assembly 16 of the blood collection set 10 includes a hub member 30, a shield member 40, and at least one extendable member 70 and, preferably, a pair of extendable members 70, 100 connecting the hub member 30 and the shield member 40. The hub member 30, shield member 40, and extendable member 70 may be integrally formed as a unitary body, which is desirably molded from a thermoplastic material. However, the hub member 30, shield member 40, and extendable member 70 may be separate parts, which are preferably molded from thermoplastic material.

The hub member 30 has a proximal end 32 and a distal end 34 and is generally defined as a rigid tubular wall 36 extending from the proximal end 32 to the distal end 34. The tubular wall 36 defines an internal passage 38 extending from the proximal end 32 to the distal end 34. The hub member 30 is adapted to support the proximal end 24 of the needle cannula 22. In particular, the needle cannula 22 is positioned within the internal passage 38 defined by the hub member 30 and extends outward from the distal end 34 of the hub member 30. Preferably, the needle cannula 22 and hub member 30 are formed as separate parts that are fixedly attached and secured through an appropriate medical grade adhesive, by direct mechanical attachment, or other similar means.

The shield member 40 is movable along the needle cannula 22 between a first proximal position or retracted position located adjacent the hub member 30, and a second distal position or extended position covering the puncture tip 28 of the needle cannula 22. The shield member 40 includes a housing 42 defining a central bore 44 having a distal opening 46 from which the puncture tip 28 extends when the shield member 40 is in the retracted position. The housing 42 of the shield member 40 extends coaxially about the needle cannula 22 and is in axial alignment with the internal passage 38 defined by the tubular wall 36 of the hub member 30. The shield member 40 further includes a pair of stabilizers in the form of wings 48, 50 that extend laterally from the housing 42 at opposing sides thereof. The lateral wings 48, 50 provide a butterfly-type wing assembly useful for positioning and placement of the needle assembly 16 and blood collection set 10 during a blood collection procedure. The shield member 40 may further include a dorsal wing 52 extending from the housing 42 and located between the lateral wings 48, 50. The dorsal wing 52 is preferably symmetrically positioned on the housing 42 between the lateral wings 48, 50.

Optionally, a needle tip guard 60 is attached to the housing 42 of the shield member 40 distally forward of the lateral wings 48, 50 and dorsal wing 52. The tip guard 60 is provided to automatically cover the distal opening 46 of the central bore 44 of the housing 42 when the shield member 40 is moved to the fully extended position covering the puncture tip 28 of the needle cannula 22. The tip guard 60 is provided as a curved leaf spring of metal or the like having an axially extending spring leg 62, a generally right-angled blocking plate 64, and a pair of clip legs 66, 68 forming a gripping collar for securely holding the tip guard 60 onto the housing 42 of the shield member 40. Operation of the tip guard 60 shown in FIGS. 1-5 will be discussed hereinafter.

As stated previously, the hub member 30 and shield member 40 are connected by at least one extendable member 70 and, preferably, a pair of extendable members 70, 100. The first extendable member 70 is comprised of a pair of folding legs, which include a first or proximal leg 72 and a second or distal leg 74. The first and second legs 72, 74 are connected by a hinged knee joint 76 that includes a fingerplate 78. The first leg 72 of the pair of folding legs is hingedly connected to the hub member 30 and the second leg 74 of the pair of legs is hingedly connected to the shield member 40. The first leg 72 includes a first end 80 and a second end 82. The first end 80 of the first leg 72 is connected to the fingerplate 78 by a hinged connection 84, and the second end 82 of the first leg 72 is connected to the hub member 30 by a hinged connection 86. Likewise, the second leg 74 includes a first end 90 and a second end 92. The first end 90 of the second leg 74 is connected by a hinged connection 94 to the fingerplate 78, and the second end 92 of the second leg 74 is connected by a hinged connection 96 to the shield member 40. Preferably, the fingerplate 78 of the knee joint 76 is concave to provide a convenient grasping location for the user of the needle assembly 16.

Similarly, the second extendable member 100 is comprised of a pair of folding legs, which include a first or proximal leg 102 and a second or distal leg 104. The first and second legs 102, 104 are connected by a hinged knee joint 106 that includes a fingerplate 108. The first leg 102 of the pair of folding legs is hingedly connected to the hub member 30 and the second leg 104 of the pair of legs is hingedly connected to the shield member 40. The first leg 102 includes a first end 110 and a second end 112. The first end 110 of the first leg 102 is connected to the fingerplate 106 by a hinged connection 114, and the second end 112 of the first leg 102 is connected to the hub member 30 by a hinged connection 116. Likewise, the second leg 104 includes a first end 120 and a second end 122. The first end 120 of the second leg 104 is connected by a hinged connection 124 to the fingerplate 108, and the second end 122 of the second leg 104 is connected by a hinged connection 126 to the shield member 40. Preferably, the fingerplate 108 of the knee joint 106 is also concave to provide a convenient grasping location for the user of the needle assembly 16. The respective folding legs 72, 74 and 102, 104 of the first and second extendable members 70, 100 are configured to generally extend laterally along the lateral sides of the needle assembly 16 when the shield member 40 is moved to the extended position.

The needle assembly 16 further includes a locking assembly 140 located on the extendable members 70, 100 for locking the shield member 40 in the extended position. In particular the locking assembly 140 is preferably integrally formed as part of the knee joints 76, 106. The locking assembly 140 is formed by a first locking member 142 and a second complimentary locking member 144. The first locking member 142 extends from the inside surface of fingerplate 108 and extends toward opposing fingerplate 78. The locking member 142 includes a sloped-leading edge 146 located opposite from a substantially planar locking edge 148. The second locking member 144 extends from the inside surface of opposing fingerplate 78 and is configured to cooperate with (i.e., receive) the first locking member 142. The locking catch member 144 also includes a sloped leading edge 150 located opposite from a substantially planar locking edge 152, which is adapted to receive the locking edge 148 formed on the first locking member 142. The operation of the locking assembly 140 will be discussed more fully hereinafter. It will be appreciated by those skilled in the art that the locations for the locking members 142, 144 may be reversed.

The needle assembly 16 further includes a compression spring 160 operatively connected between the hub member 30 and shield member 40. In particular, the compression spring 160 is connected between a proximal end 162 of the housing 42 of the shield member 40 and the distal end 34 of the hub member 30. The compression spring 160 is mechanically attached, for example by fasteners, to the proximal end 162 of the housing 42 and the distal end 34 of the hub member 30. Other equivalent means may be used to attach the compression spring 160 to the hub member 30 and shield member 40, such as by an adhesive, are within the scope of the present invention. The compression spring 160 is preferably adapted to provide outward axial forces acting on the hub member 30 and shield member 40 that continuously bias the hub member 30 and shield member 40 away from each other (i.e., axially apart). The compression spring 160 preferably continues to apply axial forces biasing the hub member 30 and shield member 40 apart when the shield member 40 is moved to its fully extended position.

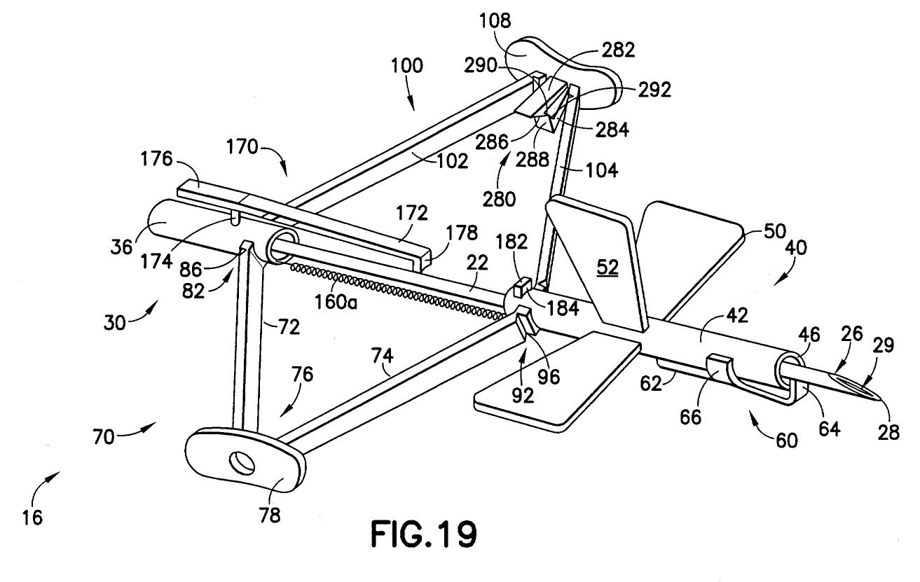

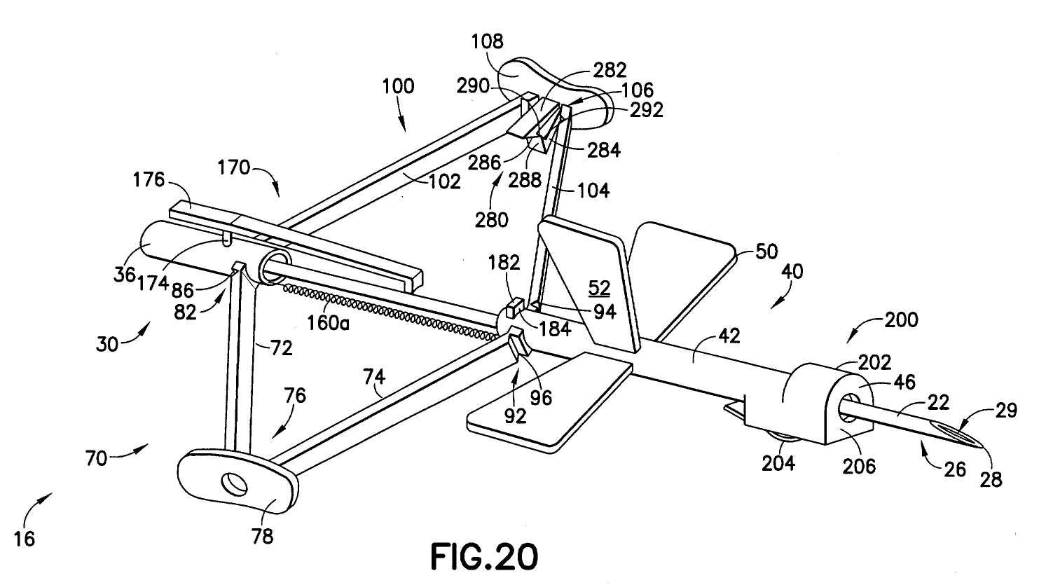

The compression spring 160 is located coaxially about the needle cannula 22. Other orientations for the compression spring 160 are also envisioned by the present invention. One such alternative orientation is shown in FIGS. 19 and 20, discussed hereinafter, in which the compression spring 160 is located adjacent and extends laterally along the needle cannula 22.

The needle assembly 16 further includes a latch assembly 170 configured to provide releasable engagement between the hub member 30 and the shield member 40. The latch assembly 170 is used to prevent the compression spring 160 from moving the shield member 40 to the extended position until the latch assembly 170 is released of engagement between the hub member 30 and the shield member 40. Once the latch assembly 170 is released of engagement between the hub member 30 and the shield member 40, the compression spring 160 provides all the necessary force to move the shield member 40 to the extended position. The latch assembly 170 includes a pivoting latch member 172 located on a top surface of the hub member 30 and extending toward the shield member 40. The pivoting latch member 172 includes a pivot support 174 about which the latch member 172 is configured to pivot. The pivotal connection between the latch member 172 and the pivot support 174 may be provided, for example, by a hinge. The latch member 172 includes a fingerplate 176 for pivoting the latch member 172 about the pivot support 174. The latch member 172 has a transversely extending edge 178 at a distal end thereof. A locking edge 180 is located opposite from the transversely extending edge 178. The latch member 172 is configured to cooperate with a rectangular-shaped catch member 182 located on the shield member 40. The catch member 182 has a locking edge 184 at a distal end thereof, which is engaged by the locking edge 180 of the latch member 172. Once the latch member 172 is in engagement with the catch member 182, the compression spring 160 will be restrained from moving the shield member 40 to the extended position. The locations for the latch member 172 and the catch member 182 may be reversed, as will be appreciated by those skilled in the art.

A removable, protective needle cover 194 may be used to cover the distal end 26 of the needle cannula 22 and, more particularly, the puncture tip 28 of the needle cannula 22. The needle cover 194 is preferably positioned over the puncture tip 28 of the needle cannula 22 in a pre-use state of the needle assembly 16, wherein the shield member 40 is in the retracted position and maintained in the retracted position by the latch assembly 170. The needle cover 194 is preferably an inexpensive, elongated plastic needle cover such as those commonly used as a needle protector in the medical field.

With the basic structure of the blood collection set 10 and needle assembly 16 now described, operation of the blood collection set 10 and needle assembly 16 will be described with reference to FIGS. 1-7. The needle assembly 16 is preferably provided with the shield member 40 in the retracted state and the needle cover 194 positioned over the distal end 26 of the needle cannula 22. The shield member 40 is held in the retracted position by the latch assembly 170. In particular, the latch member 172 extending from the hub member 30 is in engagement with the catch member 182 located on the shield member 40. The respective locking edges 180, 184 of the latch member 172 and catch member 182 maintain the engagement between the latch member 172 and the catch member 182, as well as restrain the compression spring 160 from separating the hub member 30 and the shield member 40. A blood collection tube may then be affixed to the fixture 12 located at the first end 18 of the flexible tube 14.

To use the blood collection set 10 and needle assembly 16 in a medical procedure, the user of the blood collection set 10 will first sterilize the intended area of puncture on the patient's body and remove the needle cover 194 from the distal end 26 of the needle cannula 22. The user of the needle assembly 16 may then grasp the lateral wings 48, 50 and the dorsal wing 52 to assist in positioning the needle assembly 16 at the intended area of puncture on the patient's body. The lateral wings 48, 50 and the dorsal wing 52 are preferably made flexible so that they may be folded together to provide a convenient handle for manipulating the needle assembly 16. Once the puncture tip 28 of the needle cannula 22 is inserted into a blood vessel in the patient's body (i.e., venipuncture), the user may spread the lateral wings 48, 50 flat onto the patient's body to maintain the positioning and placement of the needle assembly 16 during the blood collection procedure or other medical procedure. The lateral wings 48, 50 in this configuration will also provide a barrier between the needle cannula 22 and the user's fingertips, which will help prevent an accidental needle-stick should the needle cannula 22 inadvertently retract from the site of insertion. The user of the needle assembly 16 may also grasp the fingerplates 78, 108 attached to the extendable members 70, 100 with his or her free hand to further assist in positioning and placing the needle assembly 16 at the intended site of insertion into the patient's body.

After completing venipuncture, the user of the needle assembly 16 will typically collect one or more blood samples by attaching one or more blood collection tubes (not shown) to the fixture 12. Once the blood collection step is completed, the user of the blood collection set 10 and needle assembly 16 may then actuate the needle assembly 16 to move the shield member 40 to a partially extended position. The shield member 40 may be moved partially to the extended position while the needle cannula 22 remains in the patient's body, which significantly reduces the probability of a needle-stick wound from occurring. To actuate the needle assembly 16, the user of the needle assembly 16 pivots the latch member 172 located on the hub member 30 out of engagement with the catch member 182 located on the shield member 40 by pressing downward on the fingerplate 176.

Once the user pivots the latch member 172 out of engagement with the catch member 182, the latch assembly 170 is free of engagement between the hub member 30 and the shield member 40, and the compression spring 160 is no longer restrained. Thus, the compression spring 160 automatically (i.e., passively) moves the shield member 40 to the extended position. The compression spring 160 has a spring constant and free length sufficient to move the shield member 40 to an at least partially extended position while the needle cannula 22 is in the patient's body without entirely removing the needle cannula 22 from the patient's body. The user may then completely withdraw the needle assembly 16 from the patient's body. Thereafter, the compression spring 160 provides sufficient outward axial force between the hub member 30 and the shield member 40 to move the shield member 40 to the extended position covering the puncture tip 28 of the needle cannula 22.

In an alternative method of using the blood collection set 10 and needle assembly 16, the user may elect to entirely withdraw the needle cannula 22 from the patient's body before actuating the needle assembly 16. After completely withdrawing the needle cannula 22 from the patient's body, the user of the needle assembly 16 pivots the latch member 172 out of engagement with the catch member 182 by pressing downward on the fingerplate 176. Once the latch assembly 170 is free of engagement between the hub member 30 and the shield member 40, the compression spring 160 automatically (i.e., passively) biases the shield member 40 to the extended position covering the distal end 26 and, in particular, the puncture tip 28 of the needle cannula 22.

In either method of operation discussed hereinabove, as the shield member 40 moves forward toward its extended position, the locking members 142, 144 extending from the respective fingerplates 108, 76 of the extendable members 70, 100 begin to move toward one another. As the shield member 40 travels along the needle cannula 22 toward its extended position, the sloped leading edge 146 of the first locking member 142 slidably engages the sloped leading edge 150 of the second locking member 144. As the shield member 40 reaches its fully extended position, the locking edge 148 of the first locking member 142 snaps into engagement with the locking edge 152 of the second locking member 144 to secure the shield member 40 in the extended position. Once the locking members 142, 144 are in engagement, the extendable members 70, 100 will be prevented from moving transversely away from the lateral sides of the needle assembly 16. Thus, the locking assembly 140, once engaged, prevents the re-emergence of the puncture tip 28 of the needle cannula 22 from the shield member 40.

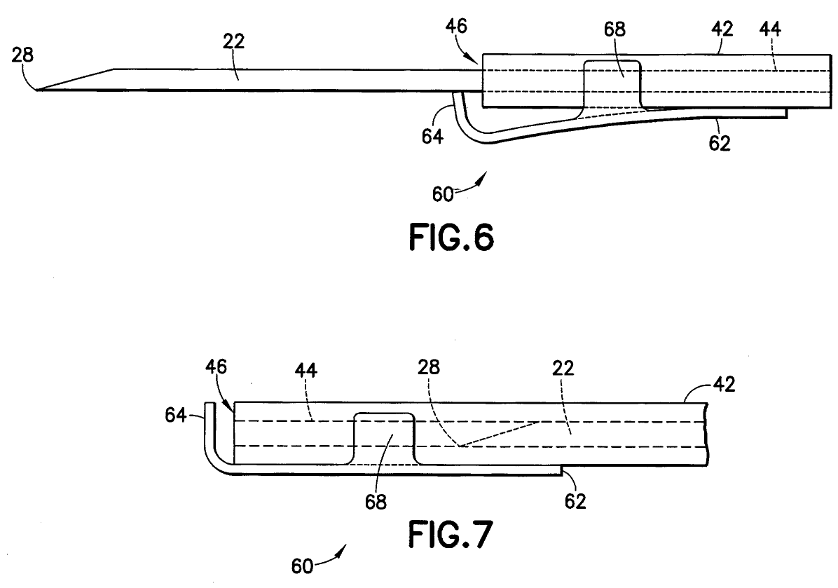

The tip guard 60 attached to the housing 42 of the shield member 40 will automatically cover the distal opening 46 of the central bore 44 once the needle cannula 22 is fully covered by the shield member 40. As shown in FIGS. 6 and 7, when the shield member 40 is in the retracted position, the needle cannula 22 extends outward from the housing 42 of the shield member 40 and the locking plate 64 of the tip guard 60 is biased into engagement with the needle cannula 22. In particular, the spring leg 62 biases the locking plate 64 into engagement with the needle cannula 22. As the needle cannula 22 is covered by the shield member 40 upon disengagement of the latch assembly 170, the locking plate 64 slides along a bottom surface of the needle cannula 22 until the needle cannula 22 is fully covered by the shield member 40. Once the locking plate 64 is no longer in engagement with the needle cannula 22, the axially extending spring leg 62 causes the locking plate 64 to automatically extend over the distal opening 46 to the central bore 44 of the housing 42. The tip guard 60 fully covers the distal opening 46 to the central bore 44 of the housing 42, thereby preventing any re-emergence of the needle cannula 22 should the latching assembly 170 inadvertently become disengaged. With the blood collection set 10 and needle assembly 16 now placed in a safety state, the blood collection tube may be safely removed from the needle assembly 16, and the needle assembly 16 disposed of as medical waste.

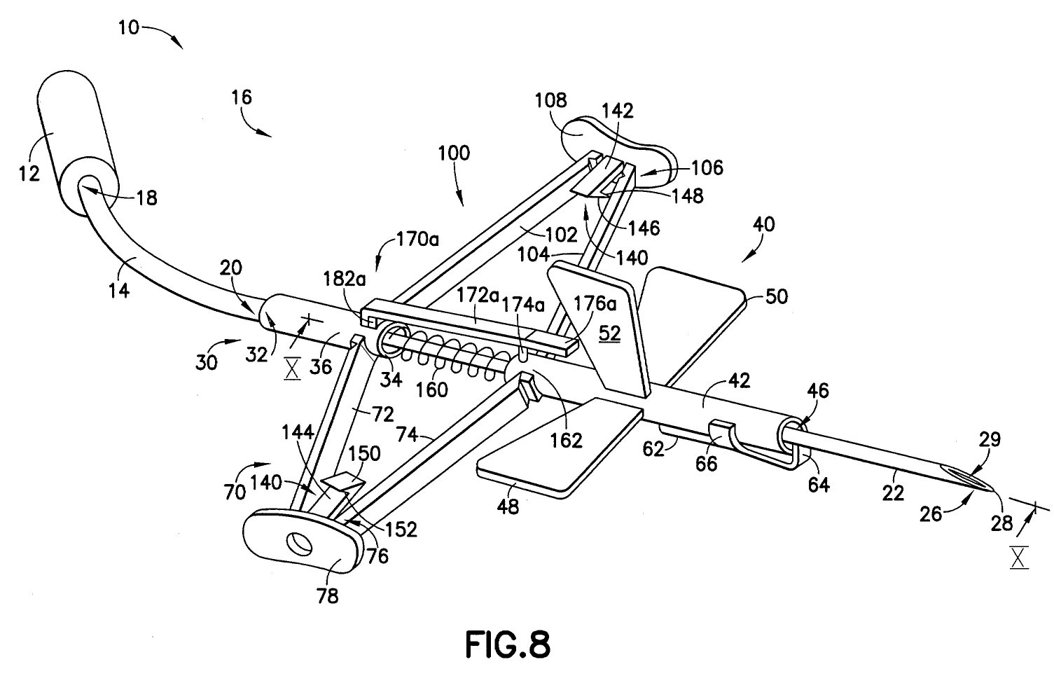

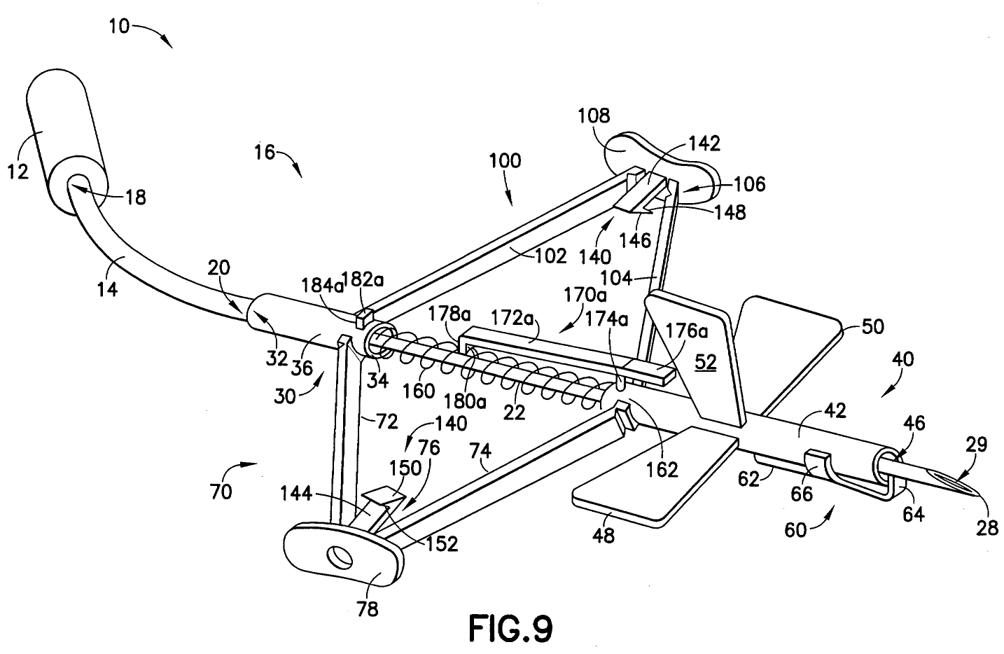

A modification to the blood collection set 10 and the needle assembly 16 as shown in FIGS. 1-5 will now be discussed with reference to FIGS. 8-10. The blood collection set 10 and needle assembly 16 shown in FIGS. 8-10 is substantially similar to the blood collection set 10 discussed in connection with FIGS. 1-5 with the modification that the locations for the latch member and the catch member of the locking assembly 170a are now reversed. In particular, as shown in FIGS. 8-10, the latch member 172a, pivot support 174a, and fingerplate 176a are now located on the top surface of the housing 42 of the shield member 40, including transversely extending edge 178a and locking edge 180a. The catch member 182a is now located on the top surface of the hub member 30. The remaining aspects and operation of the blood collection set 10 and needle assembly 16 of FIGS. 8-10 remain the same as the blood collection set 10 and needle assembly 16 discussed previously in connection with FIGS. 1-5.

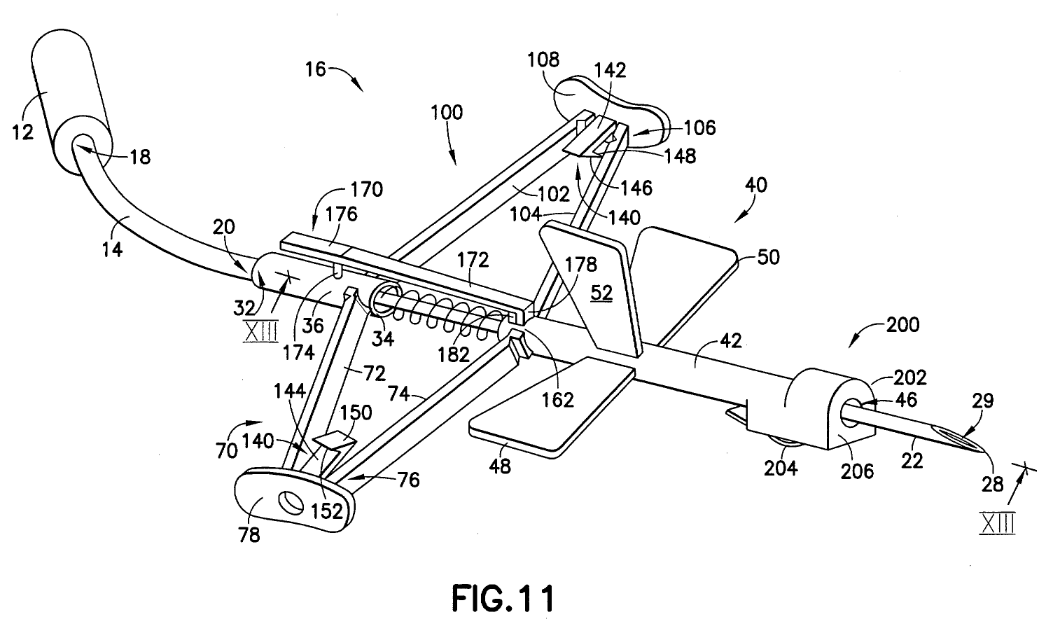

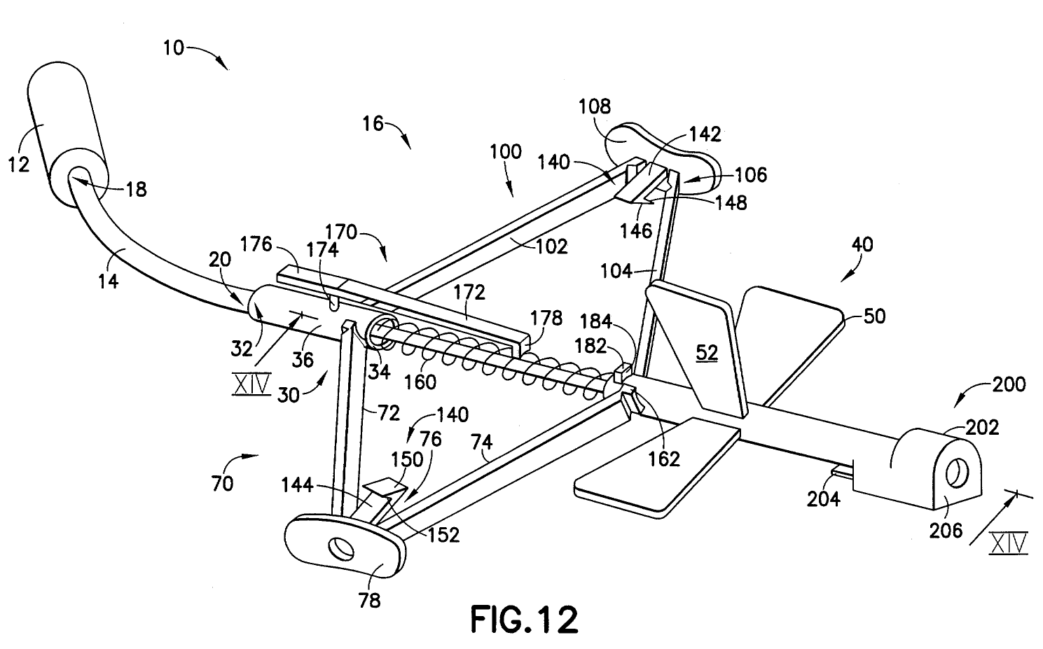

Referring to FIGS. 11-14, a further modification to the blood collection set 10 and needle assembly 16 of FIGS. 1-5 is shown. The blood collection set 10 and needle assembly 16 of FIGS. 11-15 is substantially similar to the blood collection set 10 and needle assembly 16 of FIGS. 1-5, but further includes a modified needle tip guard 200. The tip guard 200 is generally comprised of a tip guard housing 202 and a protective clip 204. The tip guard housing 202 is preferably a unitary structure molded from a thermoplastic material. The tip guard housing 202 may be formed integrally with the housing 42 of the shield member 40 as shown in FIGS. 11-14, or formed separately from and attached to the housing 42 of the shield member 40. The tip guard housing 202 includes a distal end 206, and an internal passage 208 extending through the tip guard housing 202 to cooperate with the central bore 44 of the shield member 40. Portions of the internal passage 208 adjacent the distal end 206 define an enlarged clip receptacle or recess 210. A clip mounting post 212 extends downward from the tip guard housing 202. The protective clip 204 is preferably unitarily stamped and formed from a resiliently deflectable metallic material. The protective clip 204 includes a spring leg 214 with a proximal end 216 and an opposed distal end 218. A mounting aperture (not shown) extends through the spring leg 214 at a location near the proximal end 216. The mounting aperture has a diameter approximately equal to or slightly less than the diameter of the mounting post 212 of the tip guard housing 202. As such, the mounting post 212 can be forced through the mounting aperture when the axis of the mounting post 212 and the axis of the mounting aperture are substantially co-linear. A lockout leg 220 extends angularly from the distal end 218 of the spring leg 214. The lockout leg 220 is bent back toward the proximal end 216 of the spring leg 214.

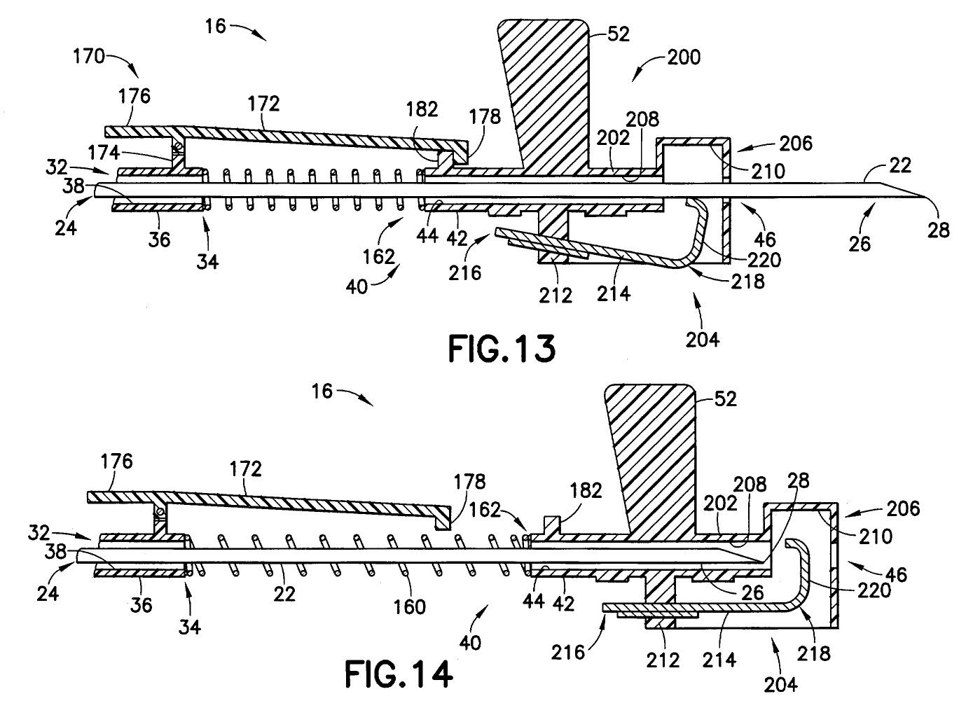

The modified tip guard 200 operates as follows. When the shield member 40 is in the retracted position, the lockout leg 220 is biased against the needle cannula 22 by the spring leg 214, as shown in FIG. 13. When the latch assembly 170 is released of engagement between the hub member 30 and the shield member 40, the compression spring 160 automatically (i.e., passively) begins to move the shield member 40 axially away from the hub member 30, as discussed previously. Simultaneously, the needle cannula 22 is withdrawn into the housing 42 of the shield member 40. During the movement of the shield member 40 axially away from the hub member 30, the lockout leg 220 remains biased in contact with the needle cannula 22 by the spring leg 214. As the shield member 40 reaches its fully extended position, the needle cannula 22 withdraws axially past the recess 210 formed by the tip guard housing 202. Because the lockout leg 220 is spring-biased toward the recess 210 by the spring leg 214, as soon as the needle cannula 22 is withdrawn past the lockout leg 220, the lockout leg 220 is spring-biased by the spring leg 214 into the recess 210. The lockout leg 220 thereby prevents re-emergence of the needle cannula 22 from the tip guard housing of the needle point guard.

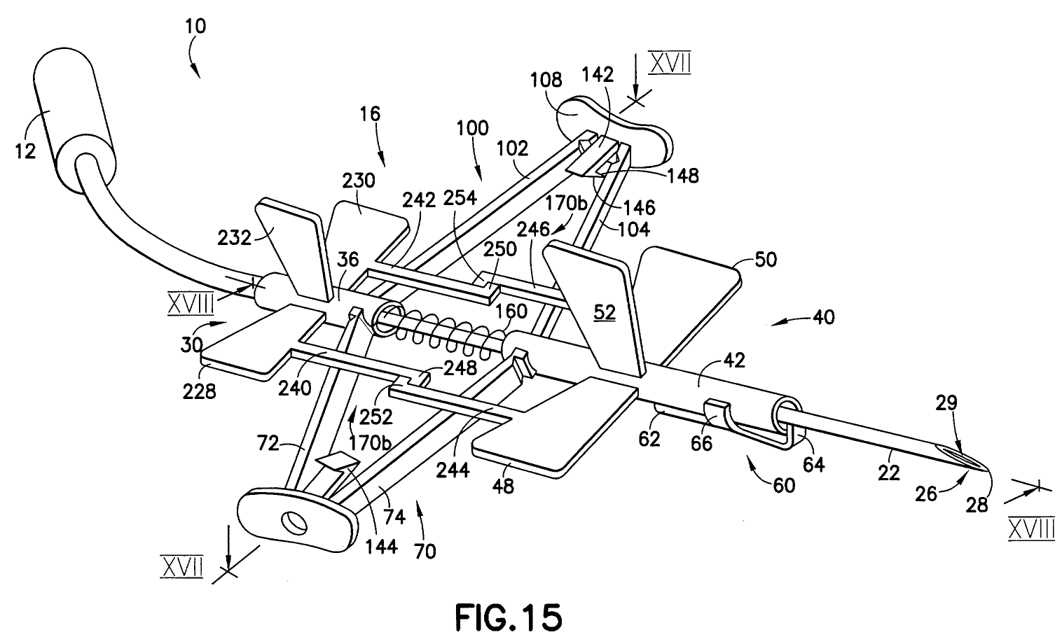

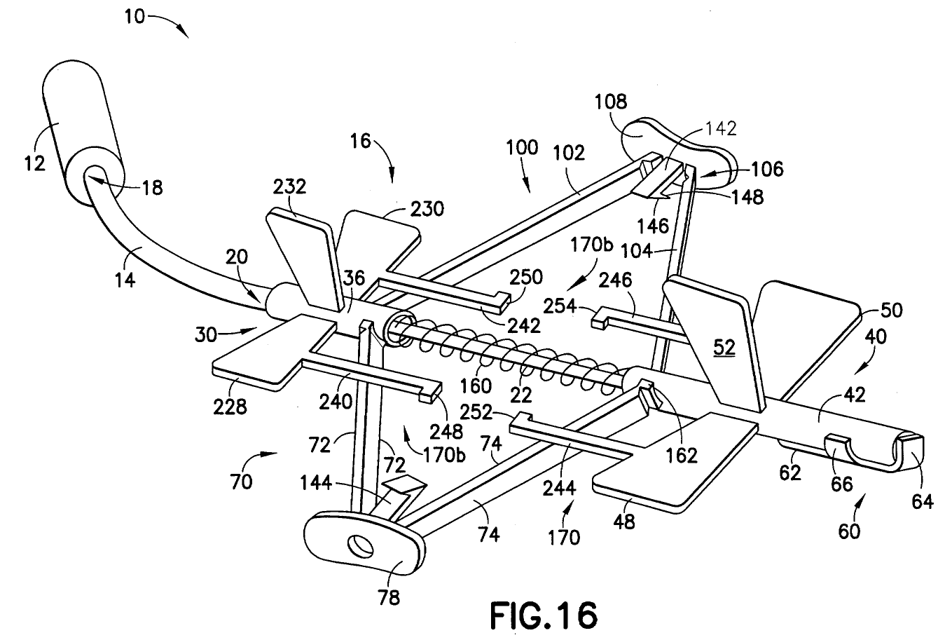

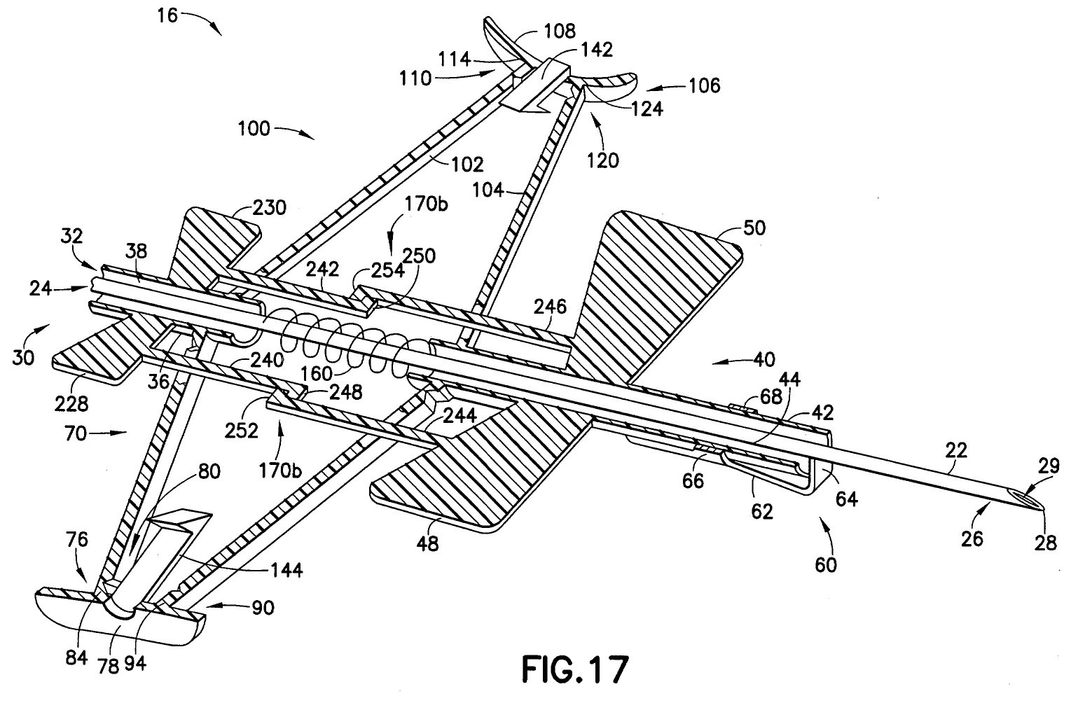

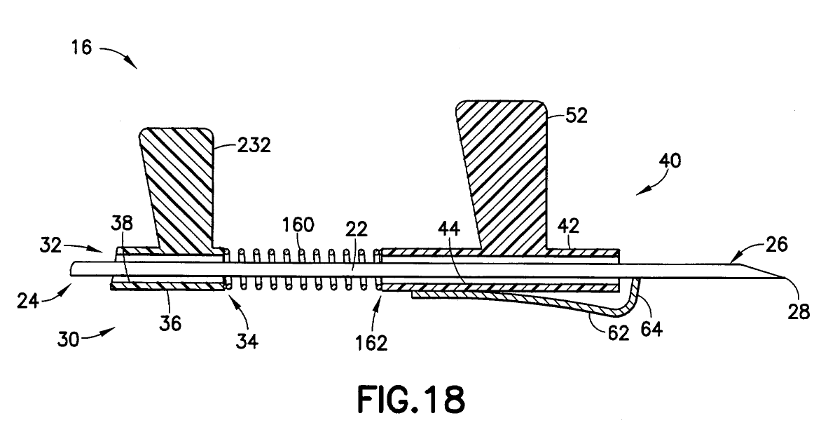

A still further modification of the blood collection set 10 and shieldable needle assembly 16 of the present invention is shown in FIGS. 15-18. The needle assembly 16 shown in FIGS. 15-18 is substantially similar to the needle assembly 16 discussed previously in connection with FIGS. 1-5 with two exceptions. First, the hub member 30 includes a pair of lateral wings 228, 230 extending symmetrically outward from the rigid tubular wall 36 of the hub member 30. A dorsal wing 232 may be located between the two lateral wings 228, 230. The dorsal wing 232 extends from a top surface of the hub member 30 and is located symmetrically between the lateral wings 228, 230. In addition, the lateral wings 228, 230 and the dorsal wing 232 are preferably made of a resiliently flexible material so that lateral wings 228, 230 and the dorsal wing 232 may be folded together in a manner similar to the lateral wings 48, 50 and the dorsal wing 52 discussed previously.

A second modification to the blood collection set 10 and needle assembly 16 shown in FIGS. 15-18 is the location and orientation of the latch assembly. The latch assembly 170b is now comprised of a pair of latch members 240, 242 located, respectively, on the lateral wings 228, 230 extending from the hub member 30. The latch members 240, 242 extend toward the lateral wings 48, 50 extending from the housing 42 of the shield member 40. The latch members 240, 242 are configured to cooperate with a second pair of latch members 244, 246 located, respectively, on the lateral wings 48, 50 extending from the housing 42 of the shield member 40. The latch members 244, 246 located on the lateral wings 48, 50 extending from the housing 42 of the shield member 40 extend proximally toward the lateral wings 228, 230 extending from the hub member 30. The latch members 240, 242 include hooked portions 248, 250 configured to mate with hooked portions 252, 254 formed on the latch members 244, 246. The respective engagement of the hooked portions 248, 250 with the hooked portions 252, 254 maintains the shield member 40 in the retracted position, and prevents the compression spring 160 from moving the shield member 40 to the extended position.

The blood collection set 10 and assembly needle 16 shown in FIGS. 15-18 may be used in a similar manner as the blood collection set 10 and needle assembly 16 discussed previously in connection with FIGS. 1-5. FIG. 15 shows the blood collection set 10 and needle assembly 16 with the shield member 40 in the retracted position. The shield member 40 is maintained in the retracted position through engagement between the respective hook portions 248, 250 and 252, 254 for the respective pairs of latch members 240, 242 and 244, 246. The latch assembly 170b is released of engagement between the hub member 30 and the shield member 40 by pivoting the lateral wings 48, 50 extending from the shield member 40 or the lateral wings 228, 230 extending from the hub member 30. This releases the engagement between the respective hook portions 248, 250 and 252, 254 for the respective pairs of latch members 240, 242 and 244, 246, which in turn releases the outward axial forces provided by the compression spring 160 to move the shield member 40 to the extended position.

Because the lateral wings 48, 50 extending outward from the shield member 40 and the lateral wings 228, 230 extending outward from the hub member 30 are made of a resiliently flexible material, the latch members 240, 242 extending from the lateral wings 228, 230 may be easily disengaged from the latch members 244, 246 extending from the lateral wings 48, 50 by pivoting the lateral wings 228, 230 upward or downward relative to the hub member 30 to release their engagement from the latch members 244, 246. A similar result occurs if the lateral wings 48, 50 are pivoted upward or downward relative to the housing 42 of the shield member 40. Additionally, the blood collection set 10 and needle assembly 16 of FIGS. 15-18 may include the tip guard 60 discussed in connection with FIGS. 1-6, or the tip guard 200 discussed in connection with FIGS. 11-14. FIGS. 15-18 illustrate the blood collection set 10 and needle assembly 16 with the tip guard 60. In all other respects, the blood collection set 10 and the needle assembly 16 shown in FIGS. 15-18 are the same as the blood collection set 10 and needle assembly 16 discussed previously in connection with FIGS. 1-5.

Referring to FIGS. 19 and 20, a further modification to the blood collection set 10 and needle assembly 16 of FIGS. 1-5 is shown. In FIGS. 19 and 20, the compression spring 160a of the needle assembly 16 is no longer located co-axially about the needle cannula 22. The compression spring 160a is now located adjacent the needle cannula 22. The compression spring 160a is connected to the shield member 40 and the hub member 30 in a similar manner as the compression spring 160a discussed previously in connection with FIGS. 1-5. The latch member 172 of the latch assembly 170 is located on the hub member 30 and the catch member 182 of the latch assembly 172 is located on the shield member 40 in a similar manner as the latch assembly 170 of FIGS. 1-5. The locations for the latch member 172 and the catch member 182 may be reversed, as discussed previously. Additionally, the blood collection set 10 and needle assembly 16 of FIGS. 19 and 20 may include the tip guard 60 discussed in connection with FIGS. 1-6, or the tip guard 200 discussed in connection with FIGS. 11-14.

The needle assembly 16 of FIGS. 19 and 20 includes a modified locking assembly 280 extending from one of the fingerplates 78, 108 toward the needle cannula 22. In FIGS. 19 and 20, the locking assembly 280 is shown extending from the inside surface of fingerplate 108. The modified locking assembly 280 is formed by a pair of locking members 282, 284. The locking members 282, 284 each have sloped edges 286, 288, respectively at distal ends thereof, which face the needle cannula 22. The locking members 282, 284 further include transversely extending locking edges 290, 292, respectively, located opposite from the sloped edges 286, 288, respectively. The locking members 282, 284 of the locking assembly 280 are preferably made of a resilient material such as molded plastic and are adapted to engage the needle cannula 22 when the shield member 40 is extended to the extended position by the compression spring 160. In particular, when the latch member 172 of the latch assembly 170 is pivoted out of engagement with the catch member 182, the compression spring 160a located adjacent the needle cannula 22 causes the shield member 40 to move axially away from the hub member 30 until the shield member 40 reaches the extended position. Simultaneously, the extendable members 70, 100 begin to move toward each other and toward the needle cannula 22. As the fingerplate 108 carrying the locking assembly 280 moves toward the needle cannula 22, the locking members 282, 284 of the locking assembly 280 begins to engage the needle cannula 22. In particular, the sloped edges 286, 288 contact and slide over the needle cannula 22, thereby separating the locking members 282, 284 to receive the shaft of the needle cannula 22. Eventually, the opposed locking members 282, 284 fully engage around the needle cannula 22, and the locking edges 290, 292 prevent the needle cannula 22 from separating from the locking assembly 280. The needle cannula 22 is now located between the locking members 252, 254. With the locking assembly 280 engaged around the needle cannula 22, the extendable members 70, 100 are prevented from extending outward away from the lateral side of the needle assembly 16, and the shield member 40 is prevented from moving from the extended position back to the retracted position adjacent the hub member 30. The tip guard 60 shown in FIG. 19 will automatically cover the distal opening 46 of the central bore 44 of the housing 42 of the shield member 40 once the shield member 40 is moved over the puncture tip 28 of the needle cannula 22. Similarly, the modified tip guard 200 shown in FIG. 20 will prevent the re-emergence of the puncture tip 28 of the needle cannula 22 once the shield member 40 and tip guard housing 202 have moved to the extended position covering the needle cannula 22.

While the blood collection set and shieldable needle assembly of the present invention have been described with respect to preferred embodiments, various modifications and alterations of the present invention may be made without departing from the spirit and scope of the present invention. The scope of the present invention is defined in the appended claims and equivalents thereto.

What is claimed is:

1. A shieldable needle assembly, comprising:
a needle cannula having a proximal end and a distal end with a puncture tip;
a hub member supporting the proximal end of the needle cannula;
a shield member having a housing defining a central bore, with the shield member in axial alignment with the hub member and extending co-axially about the needle cannula, and with the shield member movable between a retracted position in which the puncture tip of the needle cannula is exposed and an extended position in which the shield member covers the puncture tip of the needle cannula;
at least one extendable member connecting the hub member and the shield member;
a compression spring operatively connected between the hub member and the shield member; and
a latch assembly providing releasable engagement between the hub member and the shield member,
wherein when the latch assembly is in engagement between the hub member and the shield member the shield member is in the retracted position, and wherein when the latch assembly is released of engagement between the hub member and the shield member, the shield member is moved to the extended position by the compression spring, thereby covering the puncture tip of the needle cannula.

2. A needle assembly as in claim 1, wherein the latch assembly comprises a pivoting latch member located on one of the hub member and the shield member and configured to releasably engage with a catch member located on the other of the hub member and the shield member, such that when the latch member is pivoted out of engagement with the catch member, the latch assembly is released of engagement between the hub member and the shield member, thereby causing the shield member to be moved to the extended position by the compression spring.

3. A needle assembly as in claim 1, wherein the shield member includes a pair of butterfly wings extending laterally from opposing sides of the housing of the shield member.

4. A needle assembly as in claim 3, wherein the housing of the shield member further includes a dorsal wing located between the butterfly wings.

5. A needle assembly as in claim 1, wherein the hub member includes a pair of butterfly wings.

6. A needle assembly as in claim 5, wherein the hub member further includes a dorsal wing located between the butterfly wings.

7. A needle assembly as in claim 1, wherein the spring is located co-axially about the needle cannula.

8. A needle assembly as in claim 1, wherein the spring is located adjacent the needle cannula and extends between the hub member and the shield member.

9. A needle assembly as in claim 1, wherein the shield member includes a tip guard for protectively surrounding the puncture tip of the needle cannula when the shield member is moved to the extended position, and wherein the tip guard comprises a tip guard housing and a spring clip connected to the tip guard housing, with the spring clip biased against the needle cannula when the shield member is in the retracted position and resiliently extending over the puncture tip of the needle cannula when the shield member is moved to the extended position.

10. A needle assembly as in claim 1, further including a tip guard attached to the housing of the shield member and comprising a spring leg extending axially along the housing, a pair of clip legs securing the tip guard to the housing, and a locking plate resiliently biased against the needle cannula when the shield member is in the retracted position and resiliently extending over a distal opening to the central bore of the housing when the shield member is moved of the extended position for preventing the puncture tip of the needle cannula from projecting from the shield member.

11. A needle assembly as in claim 1, wherein the extendable member is comprised of a pair of folding legs connected by a hinged knee joint, with one leg of the pair of legs hingedly connected to the hub member and the other leg of the pair of legs hingedly connected to the shield member.

12. A needle assembly as in claim 11, wherein the knee joint further includes a fingerplate to enable the user to manipulate the needle assembly.

13. A shieldable needle assembly as in claim 12, further including a locking assembly for locking the shield member in the extended position, with the locking assembly comprising a pair of locking members extending from the fingerplate toward the needle cannula and configured to engage the needle cannula such that when the shield member is moved to the extended position by the compression spring, the locking members engage the needle cannula to secure the shield member in the extended position.

14. A needle assembly as in claim 12, wherein the compression spring is located adjacent the needle cannula and extends between the hub member and the shield member, and further comprising a pair of locking members extending from the fingerplate toward the needle cannula and configured to engage the needle cannula such that when the shield member is moved to the extended position by the compression spring the locking members engage the needle cannula to secure the shield member in the extended position.

15. A needle assembly as in claim 1, further comprising a second extendable member connecting the hub member and the shield member and located opposite from the first extendable member, wherein the extendable members are each comprised of a pair of folding legs connected by a hinged knee joint, with one leg of the pair of legs for each extendable member hingedly connected to the hub member and the other leg of the pair of legs for each extendable member hingedly connected to the shield member.

16. A needle assembly as in claim 15, wherein the knee joint for each extendable member includes a fingerplate.

17. A needle assembly as in claim 16, further including a locking assembly for locking the shield member in the extended position, with the locking assembly comprising a locking member extending from one of the fingerplates toward the opposing fingerplate and configured to engage a second locking member extending from the opposing fingerplate such that when the shield member is moved to the extended position by the compression spring, the locking members engage to secure the shield member in the extended position.

18. A needle assembly as in claim 1, wherein the hub member, shield member, and extendable member are integrally molded as a one-piece unit of polymeric material.

19. A needle assembly as in claim 1, wherein the hub member is configured for connection to a flexible tube of a blood collection set.

20. A needle assembly as in claim 1, further including a removable needle cover positioned over the distal end of the needle cannula.

21. A shieldable blood collection set, comprising:
a flexible tube having opposed first and second ends, with the first end of the flexible tube adapted for connection to a receptacle;
a hub member mounted to the second end of the flexible tube;
a needle cannula having a proximal end and a distal end with a puncture tip, with the proximal end connected to the hub member and the distal end projecting from the hub member, and with the needle cannula defining a lumen in fluid communication with the flexible tube and the fixture;
a shield member having a housing defining a central bore, with the shield member in axial alignment with the hub member and extending co-axially about the needle cannula, and with the shield member movable between a retracted position in which the puncture tip of the needle cannula is exposed and an extended position in which the shield member covers the puncture tip of the needle cannula;
at least one extendable member connecting the hub member and the shield member;
a compression spring operatively connected between the hub member and the shield member; and
a latch assembly providing releasable engagement between the hub member and the shield member,
wherein when the latch assembly is in engagement between the hub member and the shield member the shield member is in the retracted position, and wherein when the latch assembly is released of engagement between the hub member and the shield member, the shield member is moved to the extended position by the compression spring, thereby covering the puncture tip of the needle cannula.

22. A shieldable needle assembly, comprising:
a needle cannula having a proximal end and a distal end with a puncture tip;
a body comprising a hub member supporting the proximal end of the needle cannula; a shield member having a housing defining a central bore, with the shield member in axial alignment with the hub member and extending co-axially about the needle cannula, and with the shield member movable between a retracted position in which the puncture tip of the needle cannula is exposed and an extended position in which the shield member covers the puncture tip of the needle cannula; and a pair of extendable folding legs connecting the hub member and the shield member and extending laterally along the needle assembly, with the pair of folding legs connected by a hinged knee joint, with one leg of the pair of legs hingedly connected to the hub member and the other leg of the pair of legs hingedly connected to the shield member;
a compression spring operatively connected between the hub member and the shield member and providing an axial outward force between the hub member and the shield member; and
a latch assembly providing releasable engagement between the hub member and the shield member,
wherein when the latch assembly is in engagement between the hub member and the shield member, the shield member is in the retracted position, and wherein when the latch assembly is released of engagement between the hub member and the shield member, the shield member is moved to the extended position by the compression spring, thereby covering the puncture tip of the needle cannula.

23. A needle assembly as in claim 22, wherein both the hub member and the shield member include a pair of pivoting butterfly wings extending laterally from opposing sides thereof.

24. A needle assembly as in claim 23, wherein the shield member further includes a dorsal wing located between the butterfly wings.

25. A needle assembly as in claim 23, wherein the latch assembly comprises a pair of latch members located, respectively, on the wings extending from the shield member and configured to engage releasably with a second pair of latch members located, respectively, on the wings extending from the hub member, and wherein when the respective pairs of latch members are engaged the shield member is in the retracted position, and when one of the pairs of wings extending from the hub member and shield member are pivoted, the engaged latch members are disengaged and the shield member is moved to the extended position by the compression spring, thereby covering the puncture tip of the needle cannula.

26. A needle assembly as in claim 22, wherein the knee joint for the pair of folding legs includes a fingerplate.

27. A needle assembly as in claim 26, further including a locking assembly for locking the shield member in the extended position, with the locking assembly comprising a pair of locking members extending from the fingerplate toward the needle cannula and configured to engage the needle cannula such that when the shield member is moved to the extended position by the compression spring, the locking members engage the needle cannula to secure the shield member in the extended position.

28. A needle assembly as in claim 22, further comprising a second pair of extendable folding legs connecting the hub member and the shield member and located opposite from the first pair of extendable legs, wherein the second pair of extendable legs is connected by a hinged knee joint, with one leg of the second pair of extendable legs hingedly connected to the hub member and the other leg of the second pair of extendable legs hingedly connected to the shield member, and wherein the knee joint for the first and second pairs of extendable legs has a fingerplate.

29. A needle assembly as in claim 28, further including a locking assembly for locking the shield member in the extended position, with the locking assembly comprising a locking member extending from one of the fingerplates toward the opposing fingerplate and configured to engage a second locking member extending from the opposing fingerplate such that when the shield member is moved to the extended position by the compression spring, the locking members engage to secure the shield member in the extended position.

30. A method for automatically placing a shieldable needle assembly into a safety state in which a puncture tip of a needle cannula is covered by the needle assembly, comprising the steps of:
providing a shieldable needle assembly, comprising:
a needle cannula having a proximal end and a distal end with a puncture tip;
a hub member supporting the proximal end of the needle cannula;
a shield member having a housing defining a central bore, with the shield member in axial alignment with the hub member and extending co-axially about the needle cannula, and with the shield member movable between a retracted position in which the puncture tip of the needle cannula is exposed and an extended position in which the shield member covers the puncture tip of the needle cannula;
at least one extendable member connecting the hub member and the shield member;
a compression spring operatively connected between the hub member and the shield member; and
a latch assembly providing releasable engagement between the hub member and the shield member, with the latch assembly comprising a pivoting latch member located on one of the hub member and the shield member and engaged with a catch member located on the other of the hub member and the shield member;
using the needle assembly in a medical procedure; and
pivoting the latch member out of engagement with the catch member such that the shield member is moved to the extended position by the compression spring, thereby covering the puncture tip of the needle cannula.
